(19) United States
(12) Patent Application Publication
(10) Pub. No.: US 2026/0108597 A1
(43) Pub. Date: Apr. 23, 2026

(54) ANTIBODY MRNA FOR TREATING SARS-CORONAVIRUS-2 DELTA INFECTION AND COMPOSITION INCLUDING SAME

(71) Applicant: AGENCY FOR DEFENSE DEVELOPMENT, Daejeon (KR)

(72) Inventors: Jung Eun Kim, Daejeon (KR); Young Jo SONG, Daejeon (KR); Chi Ho YU, Daejeon (KR); Hyeongseok YUN, Daejeon (KR); Se Hun GU, Daejeon (KR); Seung Ho LEE, Daejeon (KR); Yong Han LEE, Daejeon (KR); Jiyoon SEOK, Daejeon (KR); Woong CHOI, Daejeon (KR); Suhan JUNG, Daejeon (KR); Ye Gi HAN, Daejeon (KR)

(21) Appl. No.: 19/476,218

(22) PCT Filed: Jan. 3, 2025
(86) PCT No.: PCT/KR2025/000109

§ 371 Date: Oct. 17, 2025

(30) Foreign Application Priority Data
Mar. 13, 2024 (KR) 10-2024-0034932

(51) Int. Cl. A61K 39/215; A61K 9/51; A61K 31/7105; A61K 39/00; A61P 31/14; C07K 16/104; C12N 15/85

(52) U.S. Cl. CPC A61K 39/215; A61K 9/51; A61K 31/7105; A61P 31/14; C07K 16/104; C12N 15/85; A61K 2039/53

(57) ABSTRACT

The present invention relates to an antibody mRNA for treating SARS-coronavirus-2 delta infection and a composition including same, the composition exhibiting excellent therapeutic efficacy against SARS-coronavirus-2 delta infection.

### TECHNICAL FIELD

The present disclosure relates to an mRNA antibody for treating SARS-CoV-2 Delta infection and a composition comprising the same.

### BACKGROUND ART

Coronavirus disease 2019 (COVID-19) is an infectious respiratory illness caused by severe acute respiratory syndrome coronavirus 2 (SARS-CoV-2), which presents a wide range of respiratory symptoms from mild to severe, including fever, dry cough, dyspnea, and pneumonia. In particular, patients with underlying diseases exhibit a high mortality rate, and due to the outbreak of COVID-19 that began in China in December 2019, the World Health Organization (WHO) declared a pandemic on Mar. 11, 2020.

SARS-CoV-2 is a positive-sense single-stranded RNA virus composed of approximately 30,000 nucleotides and belongs to the genus Betacoronavirus, which infects humans and animals. Under electron microscopy, it appears spherical and is surrounded by external spike proteins in a crown-like structure. The spike protein of SARS-CoV-2 is known to specifically bind to receptors on host cells.

The binding site of SARS-CoV-2 to host cells is known to be angiotensin-converting enzyme 2 (ACE2), similar to SARS-CoV. The spike protein of SARS-CoV-2 binds to human ACE2 10 to 20 times more effectively than the spike protein of SARS-CoV, and it has been reported that the receptor binding domain (RBD) of the S1 subunit of the spike protein interacts with ACE2 of host cells, thereby enabling the virus to invade into the cytoplasm of the host cells.

With the rapid spread of COVID-19, the importance of therapeutics and vaccines has been highlighted, and in particular, the need for the development of vaccines and therapeutics is increasingly emphasized to prevent and treat infectious diseases caused by new and variant viruses that could potentially be exploited as biochemical weapons.

However, as observed in the current pandemic of the SARS-CoV-2 virus, it takes at least one year to obtain product approval for vaccines and therapeutics against the virus, even under emergency use authorization. The pandemic of SARS-CoV-2, which began to spread rapidly in March 2020, reached its peak in 2021, producing numerous variants, and despite the disclosure of the SARS-CoV-2 genome sequence and the emergency use authorization of vaccines and antibody therapeutics, the pandemic could not be contained. Therefore, in the event of future outbreaks of new or variant pandemic viruses, the development of vaccines and antidotes in a radically faster manner than at present is essential.

Currently, the production of antibody therapeutics is carried out by the ‘gold standard’ method of animal cell culture and purification. These methods have the advantage of producing stable antibodies in the human body; however, in the production process of antibody therapeutics, it is critical to prevent contamination of the culture medium itself with viruses or other contaminants, which requires extensive purification steps. In addition, post-translational modifications (PTMs) must be properly formed during the in vitro production of antibodies, and since the CMC (Chemistry, Manufacturing, and Control) requirements for each of these processes are stringent, the rapid production of antibodies against diseases is challenging.

Meanwhile, to overcome these difficulties, antibody therapeutics based on mRNA are being developed. The mRNA-antibody platform allows easy delivery into the body through lipid nanoparticle (LNP) formulation, and since the antibody is directly generated after delivery in vivo, it minimizes contaminants that may arise during cell culture. Moreover, PTMs are accurately generated in vivo, which makes the CMC requirements relatively less demanding. Due to these advantages, once an mRNA-antibody candidate is selected, it can rapidly progress into clinical trials.

In the present disclosure, SARS-CoV-2 Delta spike was used as an antigen, and instead of the conventional adjuvant-protein complex for antibody generation, the antigen was prepared in the form of an mRNA vaccine and administered to animals, thereby inducing high immunogenicity with a small amount and enabling sufficient antibody production within four weeks. Using the administered mRNA vaccine, antigen-specific B cells were isolated from peripheral blood mononuclear cells (PBMCs) and splenocytes of animals, and antibodies were directly isolated therefrom, which shortened the period required for antibody acquisition. By applying the obtained antibody sequences to the mRNA-antibody platform, and through simplification of the production process by LNP formulation, the production speed was significantly accelerated. If the mRNA-based antibody expression system described herein is utilized, vaccines and antibody therapeutics against new or variant viruses can be rapidly developed and produced within 60 days.

The inventors have developed an mRNA-based antibody expression system capable of producing mRNA-antibody therapeutics at a level that enables the initiation of preclinical trials within a short period by using the mRNA antigen and mRNA-antibody platform together with antigen-specific single memory B-cell isolation. By employing this system, an mRNA-antibody therapeutic agent for treating SARS-CoV-2 Delta infection was developed and its efficacy was confirmed, thereby completing the present disclosure.

### DISCLOSURE

### Technical Problem

The purpose of the present disclosure is to provide an mRNA antibody therapeutic agent for treating SARS-CoV-2 Delta infection.

The challenges that the present disclosure is intended to solve are not limited to those mentioned above, and other challenges not mentioned will be apparent to those skilled in the art from the following description.

### Technical Solution

In order to achieve the purpose, an aspect of the present disclosure provides an mRNA antibody for treating SARS-CoV-2 Delta infection, comprising the nucleotide sequence of SEQ ID NO: 7.

In addition, another aspect of the present disclosure provides an mRNA antibody for treating SARS-CoV-2 Delta infection, comprising the nucleotide sequence of SEQ ID NO: 23.

Further, still another aspect of the present disclosure provides an mRNA antibody for treating SARS-CoV-2 Delta infection, comprising the nucleotide sequence of SEQ ID NO: 7 and the nucleotide sequence of SEQ ID NO: 23.

In some exemplary embodiments, there is provided a lipid nanoparticle (LNP) composition for treating SARS-CoV-2 Delta infection, comprising the mRNA antibody for treating SARS-CoV-2 Delta infection, the mRNA comprising the nucleotide sequence of SEQ ID NO: 7.

In some exemplary embodiments, there is provided a lipid nanoparticle (LNP) composition for treating SARS-CoV-2 Delta infection, comprising the mRNA antibody for treating SARS-CoV-2 Delta infection, the mRNA comprising the nucleotide sequence of SEQ ID NO: 23.

In some exemplary embodiments, there is provided a lipid nanoparticle (LNP) composition for treating SARS-CoV-2 Delta infection, comprising the mRNA antibody for treating SARS-CoV-2 Delta infection, the mRNA comprising the nucleotide sequence of SEQ ID NO: 7 and the nucleotide sequence of SEQ ID NO: 23.

In some exemplary embodiments, there is provided a recombinant expression vector comprising the mRNA antibody for treating SARS-CoV-2 Delta infection.

Here, the mRNA may comprise the nucleotide sequence of SEQ ID NO: 7, the nucleotide sequence of SEQ ID NO: 23, or both of the nucleotide sequence of SEQ ID NO: 7 and the nucleotide sequence of SEQ ID NO: 23.

In some exemplary embodiments, there is provided an mRNA antibody composition for treating SARS-CoV-2 Delta infection, comprising: a lipid nanoparticle (LNP) composition as described above; and a pharmaceutically acceptable carrier. In some exemplary embodiments, there is provided a method for preparing an mRNA antibody composition for treating SARS-CoV-2 Delta infection, comprising: amplifying the recombinant expression vector of claim 7; isolating and purifying mRNA from the amplified recombinant expression vector; and producing a lipid nanoparticle (LNP) comprising the mRNA.

### Advantageous Effects

The mRNA antibody for treating SARS-CoV-2 Delta infection and the composition comprising the same according to the present disclosure overcome the limitations of conventional antibody therapeutic production, such as the risk of contamination, the necessity of purification, and stringent CMC requirements for proper PTM formation. In addition, the mRNA antibody and the composition according to the present disclosure can be rapidly developed and produced, thereby exhibiting excellent therapeutic efficacy against SARS-CoV-2 Delta infection.

The effects of the present disclosure are not limited to the aforementioned effects and should be understood to include all effects that can be inferred from the configurations of the present disclosure described in the detailed description or the claims.

### DESCRIPTION OF DRAWINGS

FIG. 1 illustrates an mRNA-based antibody expression system using antigen-specific single memory B-cell isolation according to an exemplary embodiment of the present disclosure (A: process of antigen administration with LNP-antigen mRNA injection and subsequent splenocyte isolation for antigen-specific B-cell isolation, B: process of antigen-specific single memory B-cell isolation).

FIG. 2a illustrates endpoint titration to confirm antibody production after immunization with the SARS-CoV-2 Delta spike antigen according to an exemplary embodiment of the present disclosure, and FIG. 2b illustrates the measurement results of antibody production levels at different weeks after immunization with the SARS-CoV-2 Delta spike antigen according to an exemplary embodiment of the present disclosure.

FIG. 3a illustrates the results of antigen-specific single memory B-cell isolation according to an exemplary embodiment of the present disclosure, and FIG. 3b illustrates the results of verifying heavy chain and light chain fragments of antibody candidates obtained through antigen-specific single memory B-cell isolation according to an exemplary embodiment of the present disclosure.

FIGS. 4a to 4h illustrate a comparison between the sequences of antibody candidates (DW-S-Ab-01, DW-S-Ab-02, DW-S-Ab-09, DW-S-Ab-10, DW-S-Ab-11, DW-S-Ab-21, DW-S-Ab-25, and DW-S-Ab-31, respectively, in FIGS. 4a to 4h) and germline antibody sequences according to an exemplary embodiment of the present disclosure.

FIG. 5 is a graph illustrating the pharmacokinetic (PK) test results of the antibody candidate DW-S-Ab-10 administered to mice according to an exemplary embodiment of the present disclosure.

FIG. 6 shows plaque assay results (photographs) for evaluating the neutralizing activity of antibody candidates according to an exemplary embodiment of the present disclosure.

FIG. 7 is a graph illustrating the plaque assay results (number of plaques) for evaluating the neutralizing activity of antibody candidates according to an exemplary embodiment of the present disclosure.

FIG. 8 illustrates the cytopathic effect (CPE) assay results of antibody candidates according to an exemplary embodiment of the present disclosure.

FIG. 9 illustrates the results of evaluating cell viability upon treatment with antibody candidates according to an exemplary embodiment of the present disclosure.

FIG. 10a shows plaque assay results of antibody candidates as a cell efficacy evaluation according to an exemplary embodiment of the present disclosure, in which cells were pretreated with mRNA-LNP and subsequently infected with virus, and FIG. 10b shows plaque assay results of antibody candidates as a cell efficacy evaluation according to an exemplary embodiment of the present disclosure, in which cells were post-treated with mRNA-antibody medium and then cultured.

FIG. 11 illustrates the evaluation of antiviral efficacy of antibody candidates by analyzing the RdRp gene of SARS-CoV-2 using qRT-PCR results of antibody candidates according to an exemplary embodiment of the present disclosure.

FIG. 12a is a photograph showing lung tissue observations of antibody candidates and control groups as an animal efficacy evaluation according to an exemplary embodiment of the present disclosure, and FIG. 12b illustrates the results of analyzing the degree of histopathological lung injury of the lung tissues of FIG. 12a using hematoxylin and eosin (H&E) staining as an animal efficacy evaluation of antibody candidates according to an exemplary embodiment of the present disclosure.

FIG. 13 is a photograph showing plaque assay results using hamster lung tissues as an animal efficacy evaluation of antibody candidates according to an exemplary embodiment of the present disclosure.

FIG. 14 is a graph showing the qRT-PCR analysis results of SARS-CoV-2 genes in hamster lung tissues as an animal efficacy evaluation of antibody candidates according to an exemplary embodiment of the present disclosure.

### MODE FOR INVENTION

In the following description, only parts necessary for understanding the exemplary embodiments of the present disclosure will be described, and it should be noted that descriptions of other parts will be omitted within a range that does not depart from the gist of the present disclosure.

The terms or words used in the present specification and claims should not be construed as limited to their ordinary or dictionary meanings, but should be interpreted in accordance with the technical spirit of the present disclosure based on the principle that the inventors can appropriately define the concepts of the terms to explain their invention in the best way.

Accordingly, the embodiments described in the present specification and the configurations illustrated in the drawings are merely preferred embodiments of the present disclosure, and do not represent all of the technical spirit of the present disclosure. Therefore, it should be understood that various equivalents and modifications capable of replacing them at the time of filing of the present application may exist.

Hereinafter, the present disclosure will be described in detail.

mRNA Antibody for Treating SARS-CoV-2 Delta Infection

The present disclosure provides an mRNA antibody for treating SARS-CoV-2 Delta infection, comprising the nucleotide sequence of SEQ ID NO: 7.

The present disclosure provides an mRNA antibody for treating SARS-CoV-2 Delta infection, comprising the nucleotide sequence of SEQ ID NO: 23.

In addition, the present disclosure provides an mRNA antibody for treating SARS-CoV-2 Delta infection, comprising the nucleotide sequence of SEQ ID NOs: 7 and 23.

In an exemplary embodiment of the present disclosure, an antigen was prepared using the SARS-CoV-2 Delta spike sequence, formulated as LNP-mRNA, and administered to mice to obtain antibodies. In particular, by employing a method of isolating antigen-specific single memory B cells, antibodies against a novel virus could be rapidly obtained.

Human immunoglobulin genes produce antibodies through V(D)J recombination. Unlike other cells, in the development of B cells in the adaptive immune system, germline immunoglobulin genes inherited from the parental germ cells undergo rearrangement, which is referred to as somatic recombination to distinguish it from meiotic recombination. In the early development of B cells, antibody diversity is generated through somatic recombination, and when a specific antigen enters the body, somatic hypermutation occurs in the germinal center. Somatic hypermutation takes place in B cells of the germinal center under the presence of signals from activated T cells, leading to the generation of antibodies that are more selective for the antigen. Through this process, new antigen-specific antibodies are generated from germline antibody genes inherited from the parents.

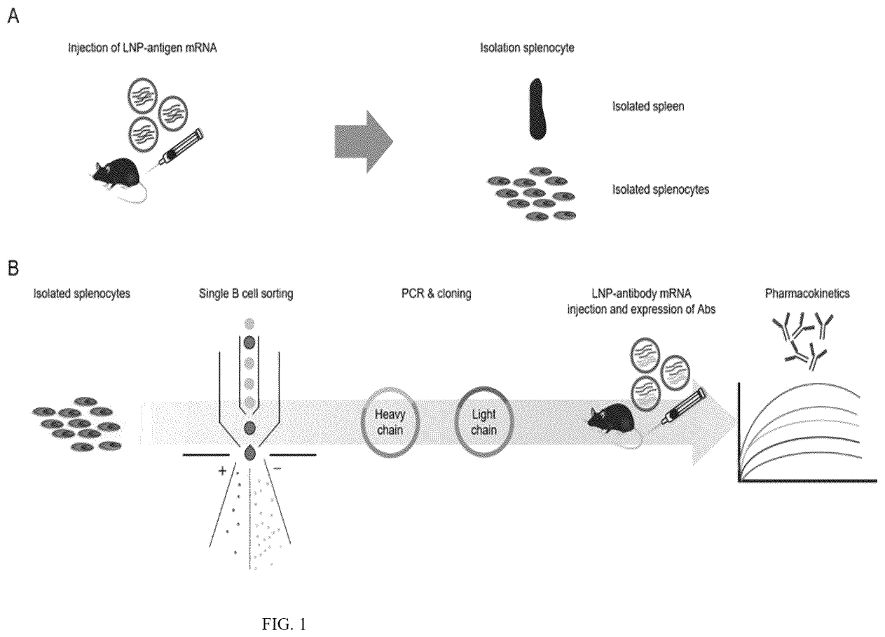

By utilizing the antibody generation mechanism through B-cell development, a system was established to develop new antibodies using a single memory B-cell isolation method (FIG. 1). First, the SARS-CoV-2 Delta spike protein was employed as an antigen, and mRNA-antigen was administered to mice as a vaccination to induce B-cell development that generates antigen-specific antibodies (FIG. 1a). The B cells possessing antigen-specific antibody sequences induced by the administered mRNA-antigen were isolated as single cells using a FACS cell sorter, and sequences of the variable regions of antigen-specific antibodies were obtained through RNA extraction from the B cells, cDNA synthesis, and PCR amplification (FIG. 1b).

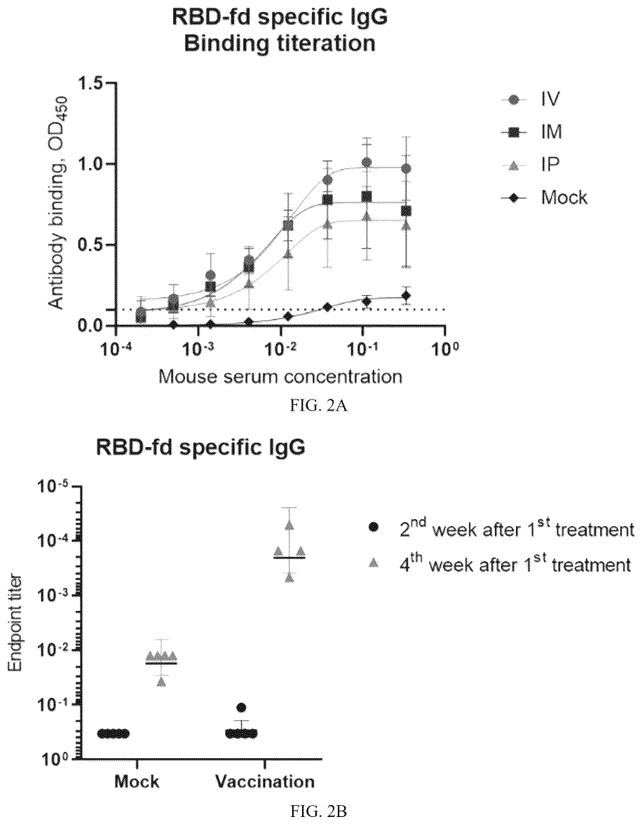

Specifically, the mRNA of the synthesized SARS-CoV-2 Delta spike antigen was formulated into LNP-mRNA, and serum obtained from mice immunized via different administration routes was analyzed to confirm antibody production. As a result, antibody generation was observed in all administration routes, and in particular, the intravenous (I.V.) route induced stronger antibody production compared to the intraperitoneal (I.P.) and intramuscular (I.M.) routes (FIG. 2a). In addition, to examine the antibody production period, antibody concentrations were measured at each week using ELISA, and it was confirmed that the highest antibody production occurred at the 4th week after immunization (FIG. 2b).

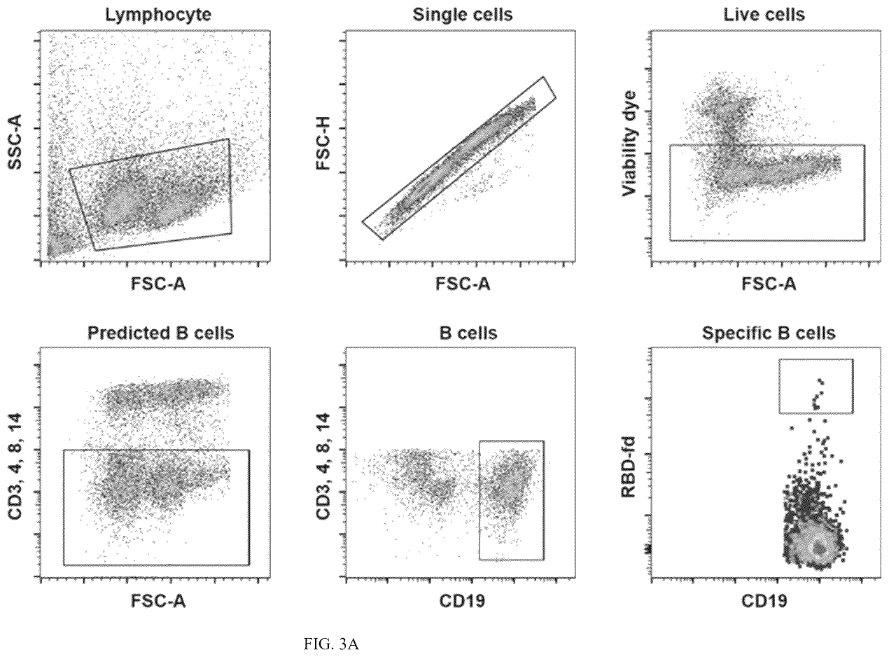

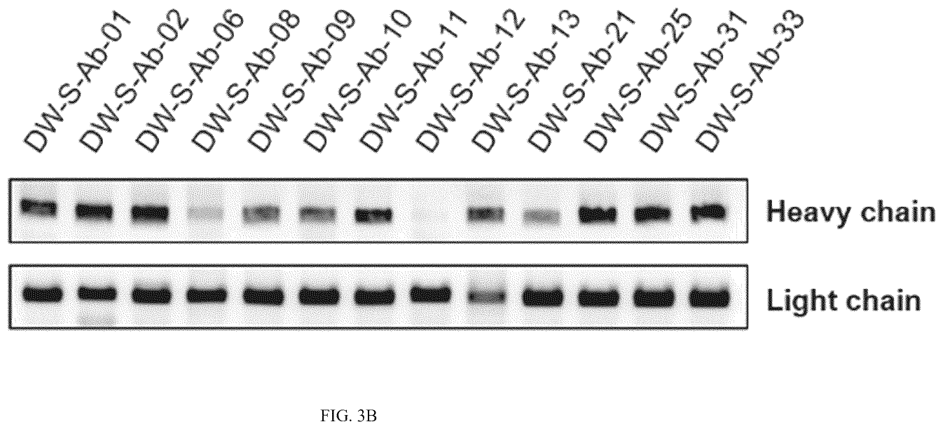

In an exemplary embodiment of the present disclosure, after intravenous (I.V.) administration of the mRNA of the SARS-CoV-2 Delta spike antigen (LNP-mRNA) to mice, spleens were excised and B cells were isolated to obtain antigen-specific B cells (FIGS. 3a and 3b), and antibody candidates without premature stop codons were ultimately obtained (Table 3).

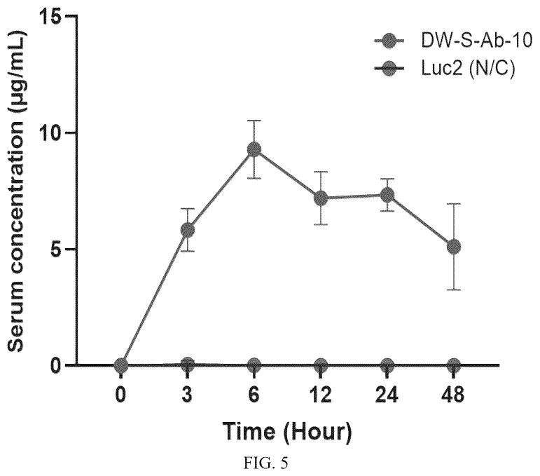

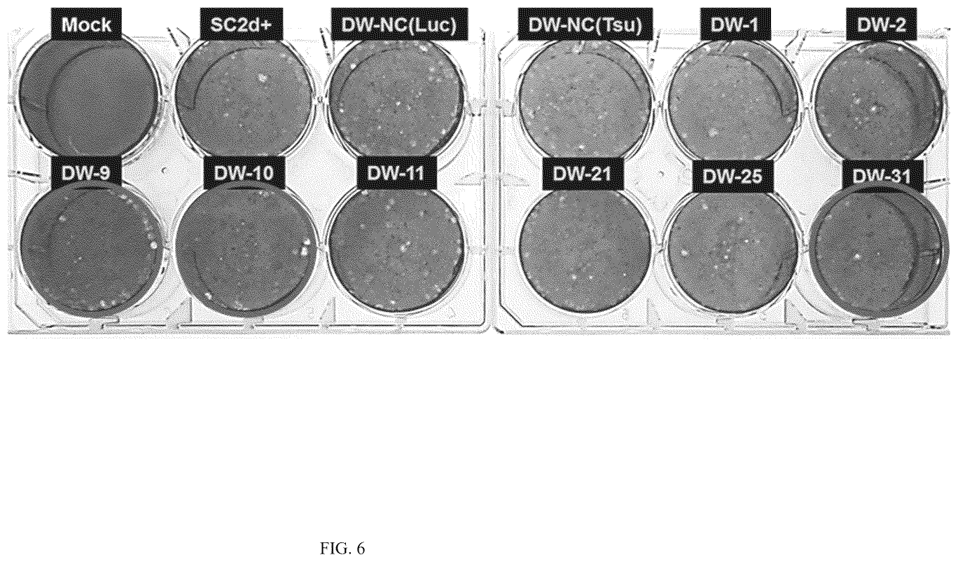

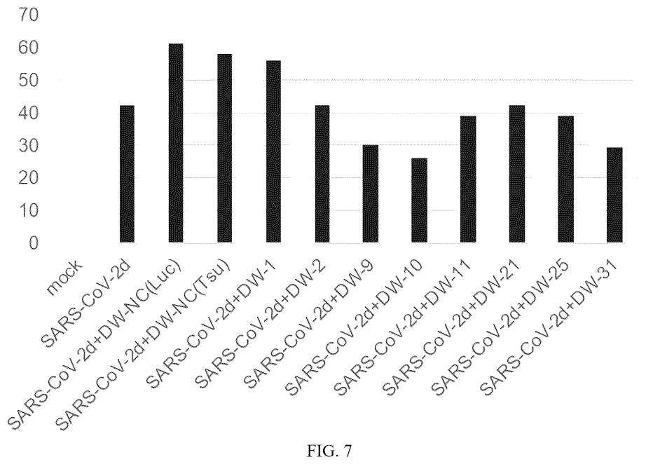

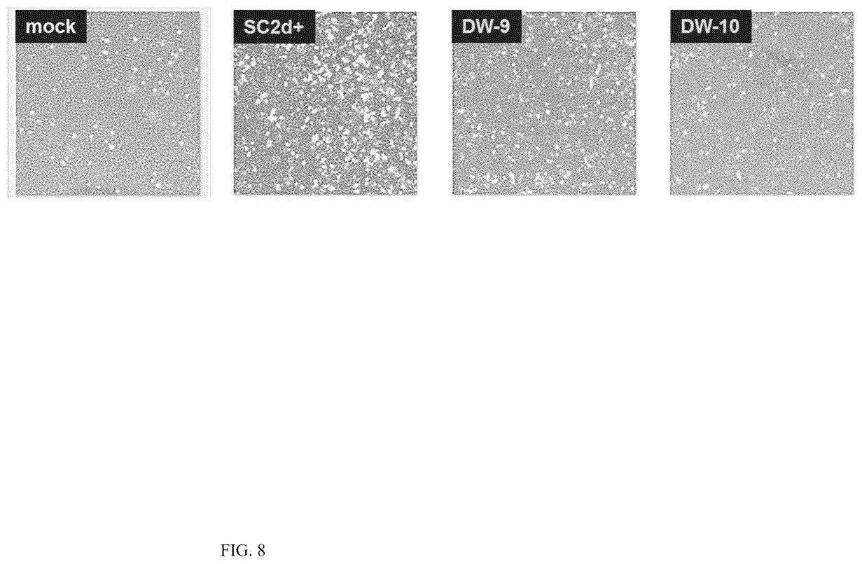

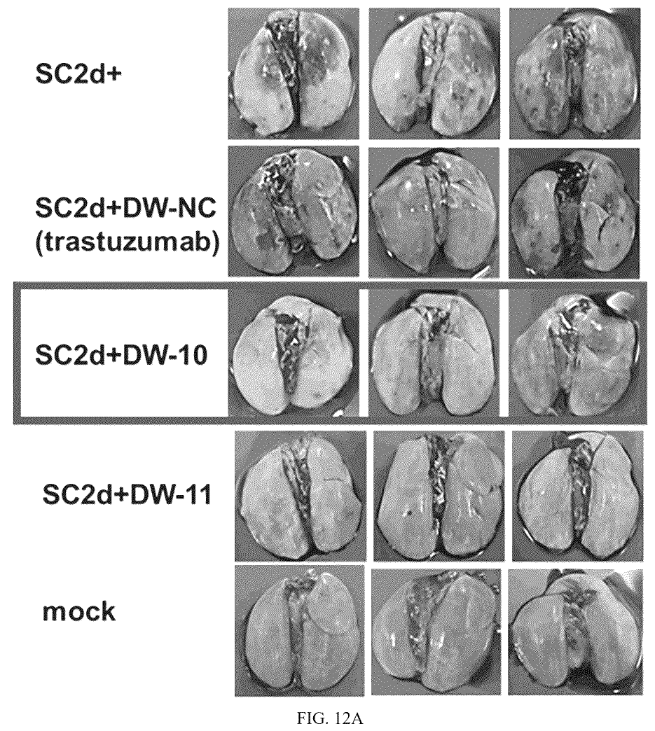

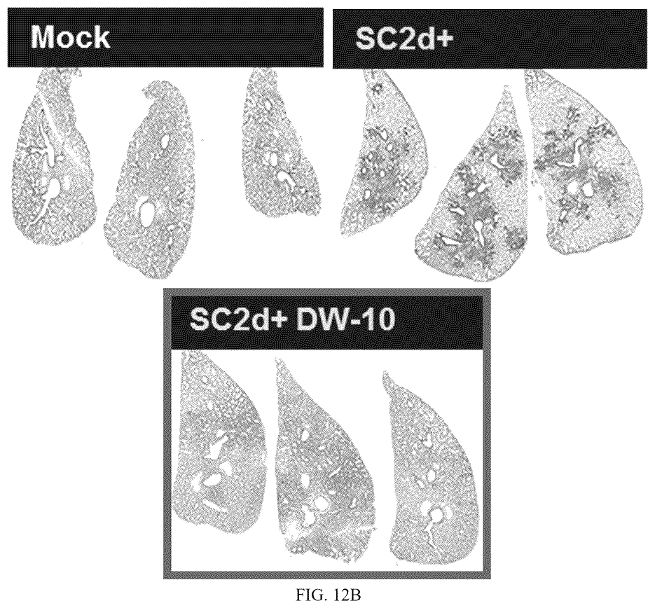

Referring to Table 3, a total of eight antibody candidates were obtained, and efficacy evaluation results demonstrated that DW-S-Ab-10 exhibited excellent efficacy in pharmacokinetic measurements (FIG. 5), antibody neutralization assays (FIGS. 6 and 7), cell efficacy evaluations (FIGS. 8, 9, 10a-10b, and 11), and animal efficacy evaluations (Table 5, FIGS. 12a-12b, 13, and 14).

### Lipid Nanoparticle (LNP) Composition for Treating SARS-CoV-2 Delta Infection

The present disclosure provides a lipid nanoparticle (LNP) composition for treating SARS-CoV-2 Delta infection, comprising an mRNA antibody comprising the nucleotide sequence of SEQ ID NO: 7.

The present disclosure provides a lipid nanoparticle (LNP) composition for treating SARS-CoV-2 Delta infection, comprising an mRNA antibody comprising the nucleotide sequence of SEQ ID NO: 23.

The present disclosure provides a lipid nanoparticle (LNP) composition for treating SARS-CoV-2 Delta infection, comprising an mRNA antibody comprising the nucleotide sequences of SEQ ID NOs: 7 and 23.

By formulating the mRNA antibody of the present disclosure into LNP-mRNA, it overcomes the innate immune response and RNA instability upon administration into the body, and enables safe delivery of the mRNA to target tissues and cells. The lipid nanoparticles comprise ionizable lipids, neutral phospholipids, cholesterol, and polyethylene glycol lipids.

In an exemplary embodiment of the present disclosure, the mRNA of the SARS-CoV-2 Delta spike antigen was formulated into LNP-mRNA, administered to mice, and spleens were excised to isolate B cells and obtain antigen-specific B cells (FIGS. 3a and 3b), and antibody candidates without premature stop codons were ultimately obtained (Table 3).

### Recombinant Expression Vector

The present disclosure provides a recombinant expression vector comprising an mRNA antibody, the mRNA antibody comprising the nucleotide sequence of SEQ ID NO: 7.

The present disclosure provides a recombinant expression vector comprising an mRNA antibody, the mRNA antibody comprising the nucleotide sequence of SEQ ID NO: 23.

The present disclosure provides a recombinant expression vector comprising an mRNA antibody, the mRNA antibody comprising the nucleotide sequences of SEQ ID NOs: 7 and 23.

The recombinant expression vector may be implemented by conventional methods known in the art of the present disclosure and is not particularly limited.

mRNA Antibody Composition for Treating SARS-CoV-2 Delta Infection

The present disclosure provides an mRNA antibody composition for treating SARS-CoV-2 Delta infection, comprising the aforementioned lipid nanoparticle (LNP) composition and a pharmaceutically acceptable carrier.

The composition comprises the aforementioned lipid nanoparticle (LNP) composition and the carrier, and the lipid nanoparticle (LNP) composition may be included as an active ingredient.

The composition may be formulated for sublingual administration, intranasal administration, or intramuscular administration, and preferably in a liquid form, for example, an injectable preparation. The liquid component may include a solvent such as water. The carrier is a pharmaceutically acceptable additive, which may be applied without limitation as long as it can be added to an antibody therapeutic composition in the technical field of the present disclosure.

Method for Preparing an mRNA Antibody Composition for Treating SARS-CoV-2 Delta Infection

The present disclosure provides a method for preparing an mRNA antibody composition for treating SARS-CoV-2 Delta infection, comprising the steps of amplifying the aforementioned recombinant expression vector, isolating and purifying mRNA from the amplified expression vector, and producing lipid nanoparticles (LNPs) comprising the mRNA.

The preparation method may allow large-scale production at high concentration and high purity for delivery efficiency and therapeutic efficacy. The method for preparing the mRNA antibody composition may employ conventional transformation processes, culture processes, isolation processes, and purification processes known in the art of the present disclosure, and is not particularly limited.

### EXEMPLARY EMBODIMENTS

Hereinafter, the present disclosure will be described in detail through exemplary embodiments. However, the following exemplary embodiments are provided only for illustrative purposes, and the scope of the present disclosure is not limited thereto.

The meanings of the terms used in the following exemplary embodiments and drawings are as follows.
Mock: Non-infected group (normal cells or animals)
SC2d+: SARS-CoV-2 Delta-infected group
DW-NC: SARS-CoV-2 Delta-infected and negative control (NC) treated group, control type=empty vector
DW-NC (Luc): SARS-CoV-2 Delta-infected and negative control (NC) treated group, control type=luciferase expression vector
DW-NC (Tsu): SARS-CoV-2 Delta-infected and negative control (NC) treated group, control type=trastuzumab expression vector

### 1. Antigen Design and Preparation

### 1-1. Experimental Animals

All animal experiments were conducted with the approval of the Institutional Animal Care and Use Committee of Daewoong Pharmaceutical (DWP-IACUC-23-041). The mice used in the experiments were purchased from Orient Bio (Seongnam-si, Gyeonggi-do, Korea). The animals were housed under controlled conditions of temperature (22±3° C.), relative humidity (55±15%), a 12-hour light/dark cycle, illumination intensity of 150-300 lx, and ventilation frequency of 10-20 times per hour. Solid feed for mice (Biopia Co., Gunpo-si, Gyeonggi-do, Korea) was provided ad libitum throughout the experimental period, and tap water was supplied ad libitum and replaced once daily.

1-2. Construction of Plasmids for Expression of SARS-CoV-2 Delta mRNA Antigen and Isolation of Single Memory B Cells

For mRNA antigen immunization in mice and single memory B-cell isolation, the sequences of SARS-CoV-2 Delta spike (GISAIS ID: EPI_ISL_2100646) and RBD-Foldon-Fc were obtained from GISAID (https://www.gisaid.org), and codon optimization was performed using the GenSmart program (https://www.genscript.com/gensmart-free-gene-codon-optimization.html) to synthesize the genes (GenScript, NJ, USA). To insert the synthesized SARS-CoV-2 Delta spike sequence and RBD-Foldon sequence into the self-constructed dual expression vector pDW202 plasmid, an oligonucleotide having the sequence 5′-AACCCACCGGTGCCACCATGTTTGTGTTCCT-3′ as a forward primer and an oligonucleotide having the sequence 5′-TGAGTGTCGACTCATCAGGGTGTAGTGCAGCTTTAC-3′ as a reverse primer were prepared for the SARS-CoV-2 Delta spike. For the RBD-Foldon, an oligonucleotide having the sequence 5′-AACCCACCGGTGCCACCATGGACTGGACCTG-3′ as a forward primer and an oligonucleotide having the sequence 5′-TGAGTGTCGACTCACTTGCCAGGGGACAGTG-3′ as a reverse primer were used to obtain amplification products. The prepared amplicons and pDW202 vector were digested with the restriction enzymes Xba I and BamH I at 37° C. for 1 hour. The amplification products were purified using a PCR purification kit (Qiagen, Venlo, Netherlands) according to the manufacturer's protocol. The digested pDW202 vector was separated and purified by agarose gel electrophoresis (1%). To ligate the purified amplicons with the pDW202 vector, a mixture containing 1 μL of Quick ligase (New England Biolabs LTD., Ipswich, MA), 1× reaction buffer, 100 ng of amplicon, and 100 ng of pDW202 vector was incubated at 25° C. for 5 minutes, followed by transformation into Escherichia coli DH5α (E. coli DH5α). The transformed E. coli DH5α were cultured on kanamycin Luria-Bertani (LB) agar plates at 37° C. for 16 hours to obtain E. coli DH5α colonies. The obtained colonies were inoculated into kanamycin-containing LB broth and cultured at 37° C. for 16 hours, followed by centrifugation to harvest the transformed E. coli DH5α. The harvested E. coli DH5α were subjected to DNA extraction using the DNA mini preparation kit (Qiagen) according to the manufacturer's protocol, thereby obtaining the SARS-CoV-2 Delta spike-pDW202 plasmid and the RBD-Foldon-Fc-pDW202 plasmid.

### 1-3. Expression and Purification of Recombinant Protein for Single Memory B-Cell Isolation

For single memory B-cell isolation, the RBD-Foldon protein was expressed. The RBD-Foldon-Fc-pDW202 plasmid was transfected into HEK293FT cells using Lipofectamine 2000 (Thermo Fisher Scientific, Waltham, MA, USA) and Opti-MEM (Thermo Fisher Scientific) according to the manufacturer's protocol.

Purification of the recombinant RBD-Foldon protein was performed using Protein G beads (GE Healthcare, Chicago, IL, USA) according to the manufacturer's protocol.

### 1-4. Preparation of Fluorescently Labeled Antigen and Isolation of Single Memory B Cells Using FACS

For antigen-specific B-cell staining, a fluorescently labeled antigen was prepared. Purified recombinant antigen (RBD-Foldon protein) was biotinylated using the EZ-Link Sulfo-NHS-LC-Biotinylation kit (Thermo Fisher Scientific) according to the manufacturer's instructions. The biotin-labeled antigen was then reacted with streptavidin-APC (Thermo Fisher Scientific) to generate a fluorescently conjugated antigen protein for antigen-specific B-cell isolation.

Antigen-specific single memory B cells were obtained from splenocytes of immunized mice. The excised splenocytes were washed twice with PBS and passed through a cell strainer to remove debris. Red blood cells were then removed using RBC lysis buffer (Thermo Fisher Scientific), and the cells were washed twice again with PBS. The cell count was adjusted to 1×10⁶ cells, and fluorescent staining was performed. The fluorescently labeled samples were incubated on ice for 20 minutes with anti-mouse CD16/32 (BD Biosciences, Franklin Lakes, NJ, USA), V450-anti-CD3 (BD Biosciences), V450-anti-CD4 (BD Biosciences), V450-anti-CD8 (BD Biosciences), V450-anti-CD14 (BD Biosciences), PE-anti-CD19 (BD Biosciences), Live/Dead staining dye (Thermo Fisher Scientific), and fluorescently labeled APC-RBD-Foldon. Among the stained cells, Live, CD3-, CD4-, CD8-, CD14-, CD19+, and RBD+ antigen-specific memory B cells were isolated using a FACSMelody Cell Sorter (BD Biosciences). The B cells were singly sorted into a 96-well plate containing buffer composed of 0.5×PBS and 0.1 U RNase inhibitor, and stored at −80° C. for subsequent reverse transcription (RT) reactions.

### 2. Antibody Generation and Production

2-1. Acquisition and Sequence Verification of Variable Regions of Novel Antibody Candidates Through cDNA Synthesis and Ig Gene Amplification

cDNA synthesis of antigen-specific single memory B cells was performed using the SuperScript III First Strand Synthesis kit (Thermo Fisher Scientific) according to the manufacturer's instructions. Ig genes were amplified using the synthesized cDNA as a template according to previously reported methods. Each amplified Ig gene product was verified and purified by electrophoresis on a 1.5% agarose gel. The purified Ig gene amplification products were cloned into the pTOP TA V2 vector using the TOPcloner™ TA core kit (Enzynomics) according to the manufacturer's instructions. The cloned Ig gene amplification products were subjected to sequencing analysis to obtain their sequences (Cosmogenetech, Seoul, Korea). The obtained sequences were analyzed using the IMGT database within IgBLAST (https://www.ncbi.nlm.nih.gov/igblast), and the germline V, D, and J gene compositions with the highest sequence homology were identified.

2-2. Construction of Plasmids for mRNA Synthesis of Novel Antibody Candidates

To synthesize the novel antibody candidates obtained through antigen-specific single memory B-cell isolation into mRNA, in vitro transcription plasmids were constructed. First, each constant region fragment of the heavy chain and light chain contained in the pFUSEss vector was amplified using the primers listed in Table 1, and the amplification products were verified and purified by gel electrophoresis. The purified fragments were digested with the restriction enzyme Xho I and then cloned into the pDW202 vector by the same method as described in Experimental Method 2, thereby constructing backbone vectors containing the constant regions of the heavy chain and light chain, respectively (HC-pDW202 and LC-pDW202).

Each novel antibody candidate fragment was designated as DW-S-Ab-01, DW-S-Ab-02, DW-S-Ab-09, DW-S-Ab-10, DW-S-Ab-11, DW-S-Ab-21, DW-S-Ab-25, and DW-S-Ab-31, and amplification products were obtained by PCR using the primers listed in Table 2. Specifically, for the heavy chain, DW-S-Ab-01 used primers 1 and 2; DW-S-Ab-02, DW-S-Ab-21, and DW-S-Ab-25 used primers 1 and 3; DW-S-Ab-09 used primers 1 and 5; DW-S-Ab-10 and DW-S-Ab-31 used primers 1 and 6; and DW-S-Ab-11 used primers 1 and 7. For the light chain, DW-S-Ab-01 used primers 8 and 9; DW-S-Ab-02 used primers 10 and 11; DW-S-Ab-09 used primers 12 and 13; DW-S-Ab-10 used primers 14 and 9; DW-S-Ab-11 used primers 8 and 11; DW-S-Ab-21 used primers 15 and 9; DW-S-Ab-25 used primers 16 and 17; and DW-S-Ab-31 used primers 8 and 17. For each of the obtained antibody candidate fragments, the variable region fragments of the heavy chains were digested with the restriction enzymes EcoR I and Nhe I, and the variable region fragments of the light chains were digested with the restriction enzymes EcoR I and BsiWI. These were then cloned into the backbone vectors to ultimately obtain in vitro transcription vectors for both heavy and light chains.

TABLE 1

| Primer name | Sequences |
|---|---|
| Backbone-HC^{a}-Forward primer | 5′-TTGCACTTGTCACGAATTCGATATCTCGAGGCTGCTAGCACCAAGGGCCCAT-3′ |
| Backbone-HC Reverse primer | 5′-GAAGCATGGCCACCGAGGCTCCAGCCTCGATCATTTACCCGGAGACAGGGA3- |
| Backbone-LC^{b}-Forward primer | 5′-TTGCACTTGTCACGAATTCGATATCTCGAGGCATCAAACGTACGGTGGCTGC-3′ |
| Backbone-LC-Reverse primer | 5′-GAAGCATGGCCACCGAGGCTCCAGCCTCGACTAACACTCTCCCCTGTTGA-3′ |

HCªHeavy chain constant region; LC^{b}Light chain constant region

TABLE 2

| | Primer name | Sequences |
|---|---|---|
| Heavy chain | Primer 1 | 5′-CTTGCACTTGTCACGAATTCGAGGTGCAGCTGCAGGAGTC-3′ |
| | Primer 2 | 5′-GATGGGCCCTTGGTGCTAGCCTGAGGAGACTGTGAGAGTG-3′ |
| | Primer 3 | 5′-GATGGGCCCTTGGTGCTAGCTGCAGAGACAGTGACCAGAG-3′ |
| | Primer 4 | 5′-GATGGGCCCTTGGTGCTAGCTGAGGAGACGGTGACCGTGG-3′ |
| | Primer 5 | 5′-GATGGGCCCTTGGTGCTAGCTGAGGAGACTGTGAGAGTGG-3′ |
| | Primer 6 | 5′-GATGGGCCCTTGGTGCTAGCTGCAGAGACAGTGACCAGAG-3′ |
| | Primer 7 | 5′-GATGGGCCCTTGGTGCTAGCTGCAGAGACAGTGACCAGAG-3′ |
| Light Chain | Primer 8 | 5′-CTTGCACTTGTCACGAATTGGATATTGTGATCACCCAGTC-3′ |
| | Primer 9 | 5′-GATGGTGCAGCCACCGTACGTTTGATTTCCAGCTTGGTGG-3′ |
| | Primer 10 | 5′-CTTGCACTTGTCACGAATTCGACTTATGTATCCTGGTATCA-3′ |
| | Primer 11 | 5′-GATGGTGCAGCCACCGTACGTTTCAGCTCCAGCTTGGTCC-3′ |
| | Primer 12 | 5′-CTTGCACTTGTCACGAATTCGGATATTGTGCTCACCCAGTC-3′ |
| | Primer 13 | 5′-GATGGTGCAGCCACCGTACGTTTTATTTCCAGCTTGGTCC-3′ |
| | Primer 14 | 5′-CTTGCACTTGTCACGAATTCGGACATTGTGATCACCCAGAC-3′ |
| | Primer 15 | 5′-CTTGCACTTGTCACGAATTCGGACATTGTGATGACACAGTC-3′ |
| | Primer 16 | 5′-CTTGCACTTGTCACGAATTCGGACATTGTGCTCACCCAGTC-3′ |
| | Primer 17 | 5′-GATGGTGCAGCCACCGTACGTTTTATTTCCAGCTTGGTCC-3′ |

2-3. Antigen and Novel Antibody Candidate mRNA Synthesis

To obtain mRNA through in vitro transcription, the SARS-CoV-2-delta-Spike-pDW202 plasmid was linearized by digestion with the restriction enzyme Sap I. The linearized plasmid was purified using the DNA Maxi preparation kit (Qiagen) according to the manufacturer's instructions. For the synthesis of SARS-CoV-2-delta-Spike mRNA, a 400 μL reaction mixture containing 20 μg of the linearized plasmid, 8 mM of AG Cap (Trilink, San Diego, CA, USA), 10 mM of each rNTP, 1× reaction buffer, and 40 μL of MEGAscript T7 enzyme mix (Thermo Fisher Scientific) was incubated at 37° C. for 4 hours. Subsequently, 40 μL of DNase I (Thermo Fisher Scientific) was added, and the reaction was carried out at 37° C. for 15 minutes, followed by purification of the synthesized mRNA using the LiCl purification method.

### 2-4. LNP-mRNA Formulation

For LNP-mRNA formulation, all preparations were first dissolved in ethanol at molar ratios of 50% ionizable lipid, 38.5% cholesterol, 10% DSPC, and 1.5% PEG2000-DMG. For the mRNA of novel antibody candidates, the heavy-chain and light-chain mRNAs were mixed at a 1:1 molar ratio. The lipid mixture was then combined with an mRNA aqueous solution in 0.1 M sodium citrate buffer (pH 4.5) using a microfluidic mixing cartridge (NanoAssemblr Ignite cartridges, Precision Nanosystems Inc., Vancouver, Canada) under conditions of an N/P (amine-to-phosphate) ratio of 6 and an ethanol-to-water ratio of 1:3. The resulting mixture was dialyzed against PBS (pH 7.4) with a volume at least 10,000-fold greater than the sample for one day (24 hours). The particle size of the formulated LNP-mRNA was measured by dynamic light scattering (DLS) using a Malvern Zetasizer Nano ZS.

### 2-5. LNP-mRNA Vaccination

For novel antibody discovery, six-week-old Balb/C mice were administered with 0.5 mpk of LNP-mRNA antigen vaccine via intravenous (I.V.), intramuscular (I.M.), or intraperitoneal (I.P.) injection routes. An additional dose of 0.5 mpk was administered through the same routes two weeks later, followed by another 0.5 mpk dose on day 25. Blood samples were collected weekly by venipuncture, with whole blood collected at week 4. Serum was obtained by centrifugation at 10,000 rpm for 30 minutes at 4° C. The antibody concentration in serum samples was measured using a Human IgG ELISA Kit (Thermo Fisher Scientific) according to the manufacturer's protocol.

Using weekly serum samples, endpoint dilution titration assays were performed by ELISA to identify animals that generated antibodies (FIGS. 2a-2b). The results confirmed antibody generation across all administration routes, with the I. V. route producing superior antibody responses compared to the I.P. and I.M. routes (FIG. 2a). Furthermore, ELISA-based monitoring of weekly antibody titers revealed that the highest antibody levels were observed at week 4 after vaccination (FIG. 2b).

2-6. Isolation of Novel Antibody Candidates through Antigen-Specific Single Memory B Cell Sorting

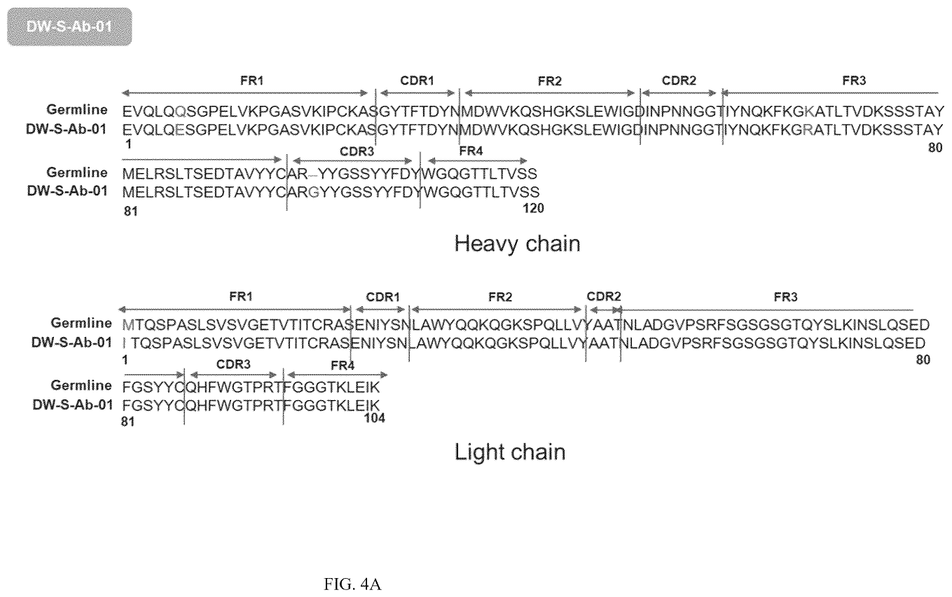

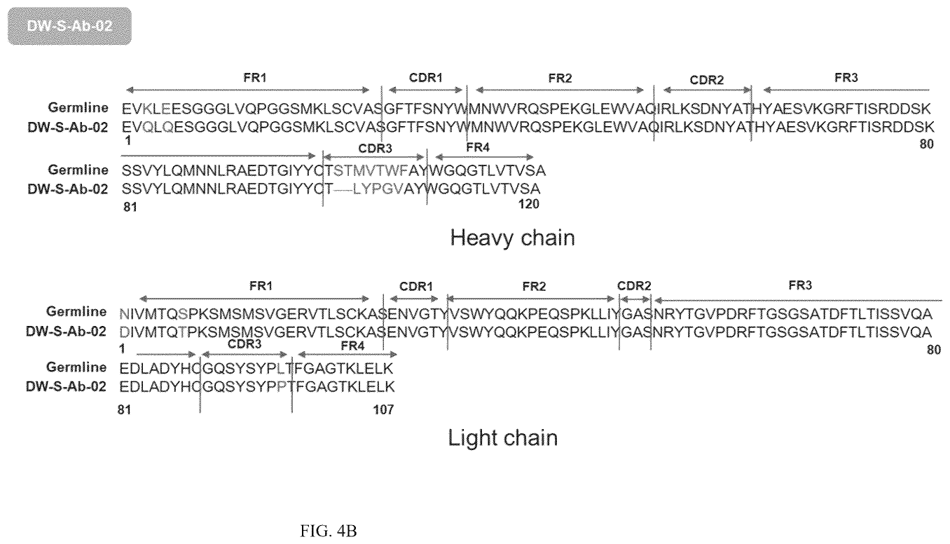

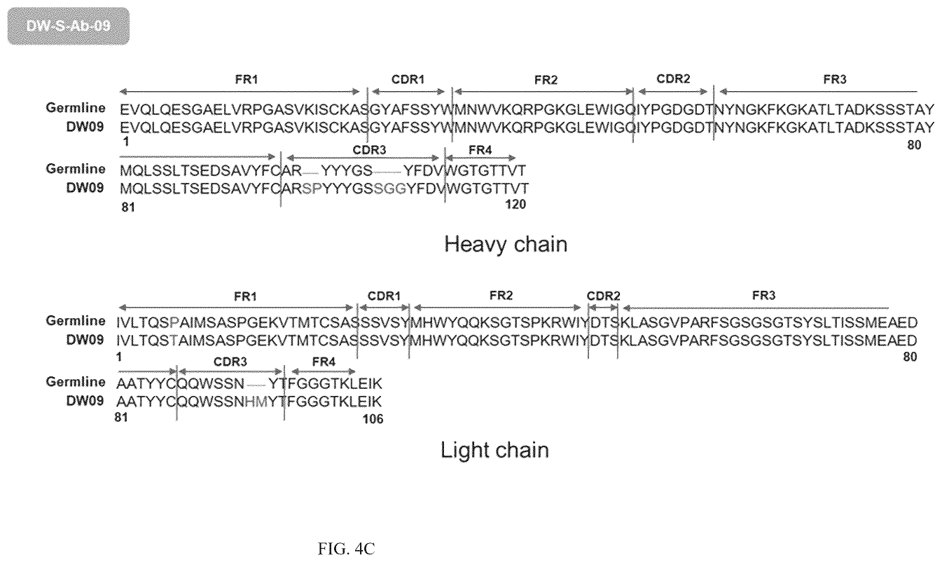

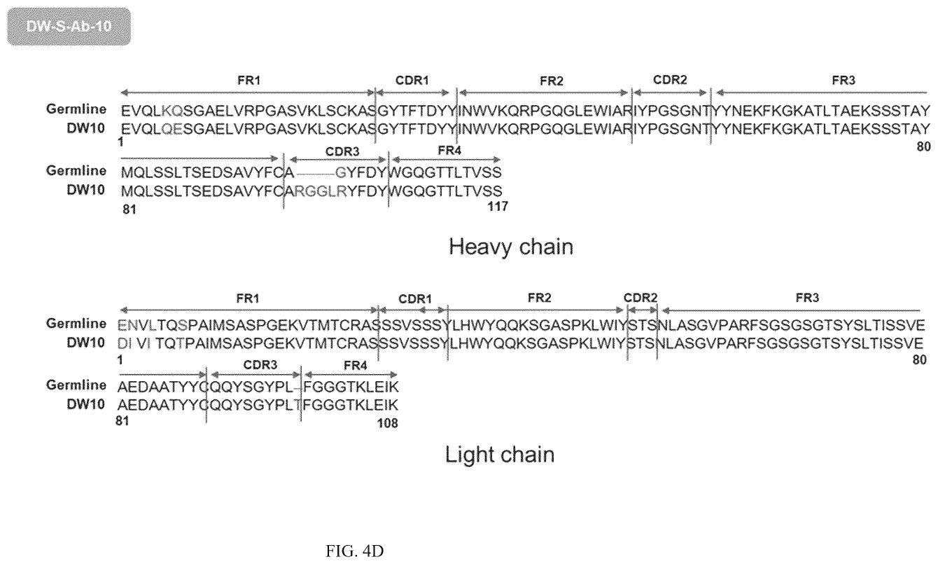

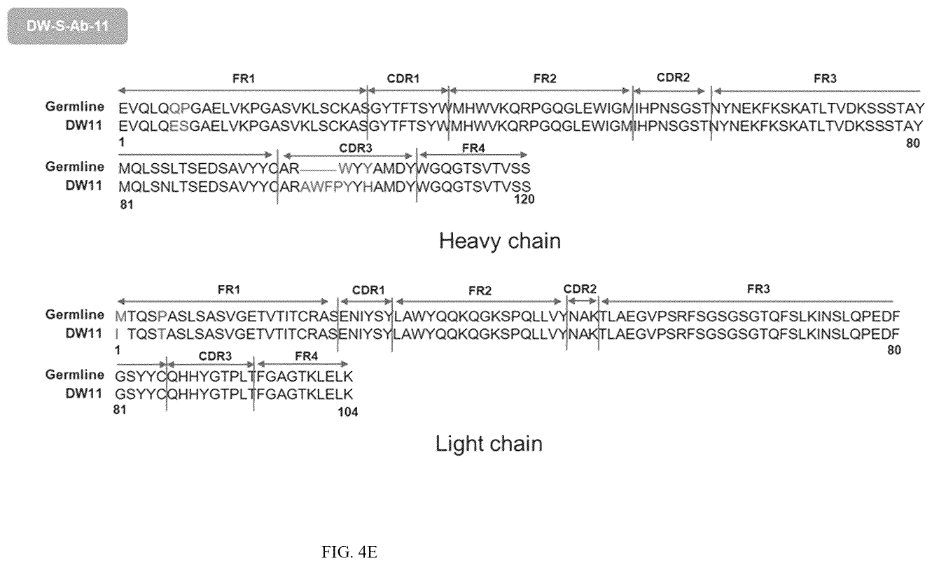

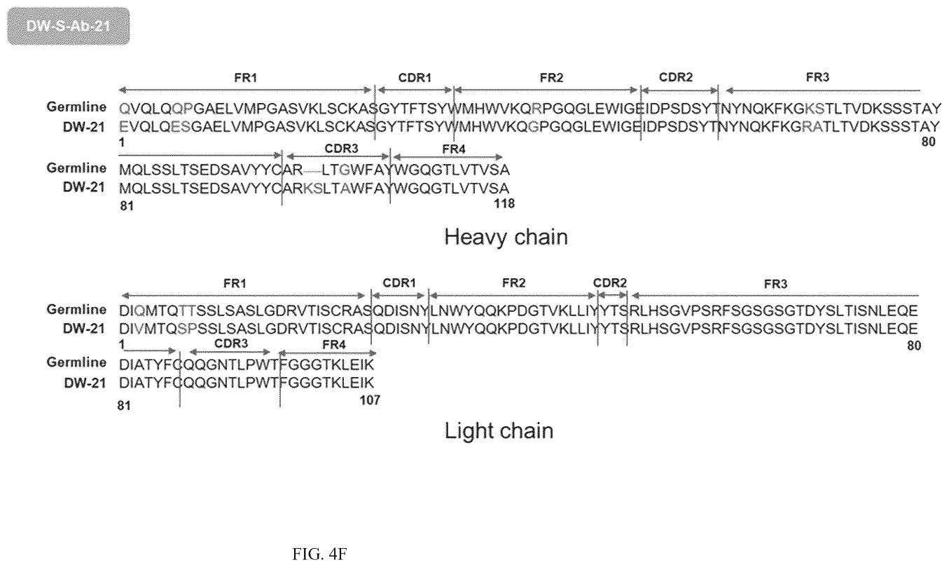

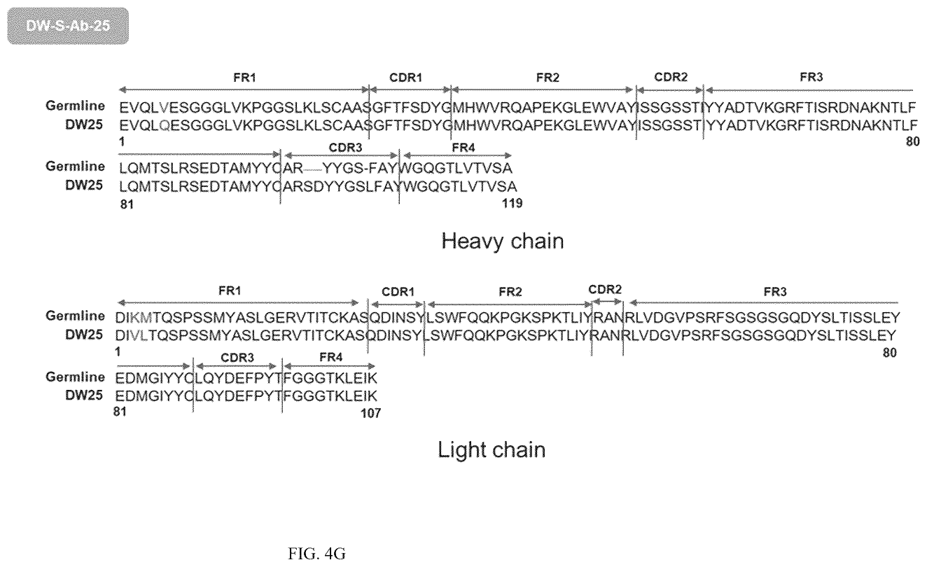

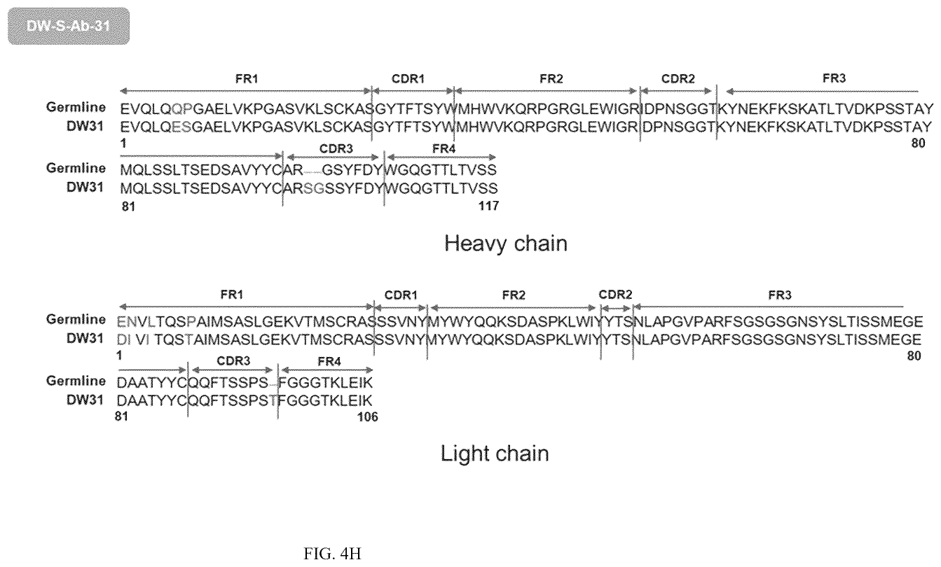

To obtain novel antibody candidates, C57BL/6 mice were intravenously (I. V.) immunized with LNP-SARS-CoV-2-delta-Spike-mRNA, and spleens were harvested at week 4. Approximately 5,000 B cells were initially isolated through LIVE/DEAD staining, followed by negative selection of CD4-, CD8-, and CD14-specific B cells. Subsequently, CD19+/RBD-foldon-APC+ antigen-specific B cells were sorted, resulting in the isolation of approximately 16 antigen-specific B cells (FIG. 3a). RNA extracted from these 16 B cells was subjected to reverse transcription PCR (RT-PCR) and nested PCR, yielding 13 antibody sequences (FIG. 3b). Sequence analysis of these antibodies revealed that 8 complete antibody candidates without premature stop codons were successfully obtained (Table 3). Further germline mutation analysis of the 8 antibody candidates demonstrated significant variation in the CDR3 region, which constitutes the antigen-binding site (Table 4, FIGS. 4a-4h). Since the CDR3 region directly influences antigen-binding affinity, these observed variations were presumed to enhance antigen specificity.

TABLE 3

| Antibody | Heavy chain | | | Light chain | |
|---|---|---|---|---|---|
| candidates | V | D | J | V | J |
| DW-S-Ab-01 | IGHV1-18*01 | IGHD1-1*01 | IGHJ2*02 | IGKV12-46*01 | IGKJ1*01 |
| DW-S-Ab-02 | IGHV6-3*01 | IGHD2-1*01 | IGHJ3*01 | IGKV6-20*01 | IGKJ5*01 |
| DW-S-Ab-09 | IGHV1-80*01 | IGHD1-1*01 | IGHJ1*03 | IGKV4-50*01 | IGKJ2*01 |
| DW-S-Ab-10 | IGHV1-76*01 | IGHD4-1*01 | IGHJ2*01 | IGKV4-57-1*01 | IGKJ1*01 |
| DW-S-Ab-11 | IGHV1-64*01 | IGHD2-2*01 | IGHJ4*01 | IGKV12-44*01 | IGKJ5*01 |
| DW-S-Ab-21 | IGHV1-69*01 | IGHD4-1*01 | IGHJ3*01 | IGKV10-96*01 | IGKJ1*01 |
| DW-S-Ab-25 | IGHV5-17*01 | IGHD1-1*01 | IGHJ3*01 | IGKV14-111*01 | IGKJ2*01 |
| DW-S-Ab-31 | IGHV1-72*01 | IGHD1-1*01 | IGHJ2*01 | IGKV4-50*01 | IGKJ2*01 |

TABLE 4

| Antibody | Germline Sequence | | Novel Sequence | |
|---|---|---|---|---|
| candidates | Heavy chain | Light chain | Heavy chain | Light chain |
| DW-S-Ab-01 | ARYYGSSYYFDY | QHFWGTPWT | ARG^{a}YYGSSYYFDY | QHFWGTPRT |
| DW-S-Ab-02 | TSTMVTWKAY | GWSYSYPLT | T^{b}LYPGVAY | GQSYSYPPT |
| DW-S-Ab-09 | ARFTTTVVAYWYFDV | QQWSSNPPVT | ARS^{a}PYYYGSSGGYFDV | QQWSSNHMYT |
| DW-S-Ab-10 | ARLTGYFDY | QQYSGYFLW | ARG^{a}GLRYFDY | QQYSGYPLT |
| DW-S-Ab-11 | ARSTMVTVYAMDY | QHHYGTPLT | ARAWFPYYHAMDY | QHHYGTPLT |
| DW-S-Ab-21 | ARLTGWFAY | QQGNTLPWT | ARKS^{a}LTAWFAY | QQGNTLPWT |
| DW-S-Ab-23 | ARFTTTVVAWFAY | LQYDEFPYT | AR^{b}SDYYGSLFAY | LQYDEFPYT |
| DW-S-Ab-31 | ARFTTTVVAYFDY | QQFTSSPSYT | ARSGS^{b}SYFDY | QQFTSSPS^{b}T |

ªindicates the insertion site, ^{b}indicates the deletion site, and the letters marked in red

### 3. Efficacy Evaluation

### 3-1. Pharmacokinetics (PK) Measurement of Novel Antibody Candidates

To evaluate the in vivo expression level of the mRNA-novel antibody candidates, pharmacokinetic experiments were conducted. Six-week-old male C57BL/6 mice (Orientbio) were administered 2 mpk of the mRNA-novel antibody via tail vein injection. Blood samples were collected by intravenous blood collection at 0 h, 3 h, 6 h, 12 h, 24 h, and 48 h post-injection. Serum was obtained by centrifugation of the blood at 10,000 rpm for 30 minutes at 4° C. The antibody concentration in each serum sample was measured using a Human IgG ELISA Kit (Thermo Fisher Scientific) according to the manufacturer's instructions.

The pharmacokinetic analysis of antibody candidate DW-S-Ab-10 demonstrated that antibody expression peaked at 6 hours post-injection and maintained a concentration of approximately 5 μg/ml for up to 48 hours (see FIG. 5).

### 3-2. Antibody Neutralization Evaluation

All efficacy evaluation experiments using SARS-CoV-2 delta were conducted under the approval of the Institutional Biosafety Committee of the Agency for Defense Development (ADD-IBC-2022-2-02) and the Institutional Animal Care and Use Committee (ADD-IACUC-22-16), in biosafety level 3 (BSL-3 and ABL-3) facilities.

The neutralization capacity of eight antibody candidates, including DW-S-Ab-10, was evaluated using a plaque assay with Vero E6 cells. For the assay, the mRNA-antibody medium (mRNA: Ab medium, defined as the culture medium containing antibodies secreted by cells transfected with antibody candidate mRNA) was mixed with SARS-CoV-2 delta at a 1:1 ratio and incubated at room temperature for 1 hour. The mixture was then used to infect Vero E6 cells. After 1 hour of infection at 37° C. in a CO₂ incubator, the inoculum was removed, the cells were washed once with PBS, and an agar overlay was applied. The cells were further incubated at 37° C. in a CO₂ incubator for 3 days. At 72 hours post-infection, cells were fixed with 4% paraformaldehyde (PFA), the agar overlay was removed, and the cells were stained with 0.2% crystal violet to perform a plaque assay.

The neutralization assay of the eight mRNA-antibody candidates revealed that, compared to the SARS-CoV-2 delta infection group, DW-S-Ab-9, DW-S-Ab-10, and DW-S-Ab-31 exhibited reduced plaque numbers, indicating superior neutralization capacity (FIG. 6). Among these, DW-S-Ab-10 demonstrated the strongest neutralization activity (FIG. 7).

### 3-3. Cellular Efficacy Evaluation

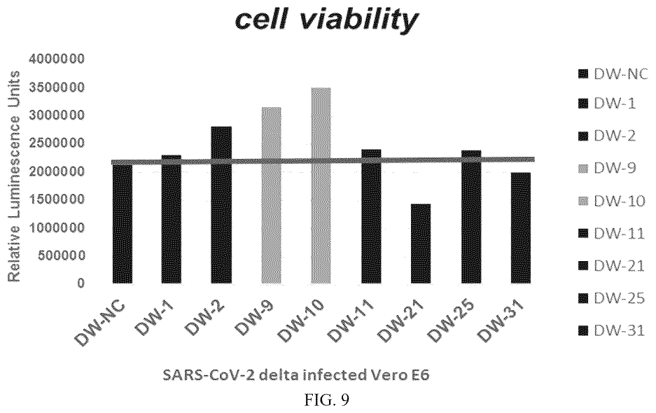

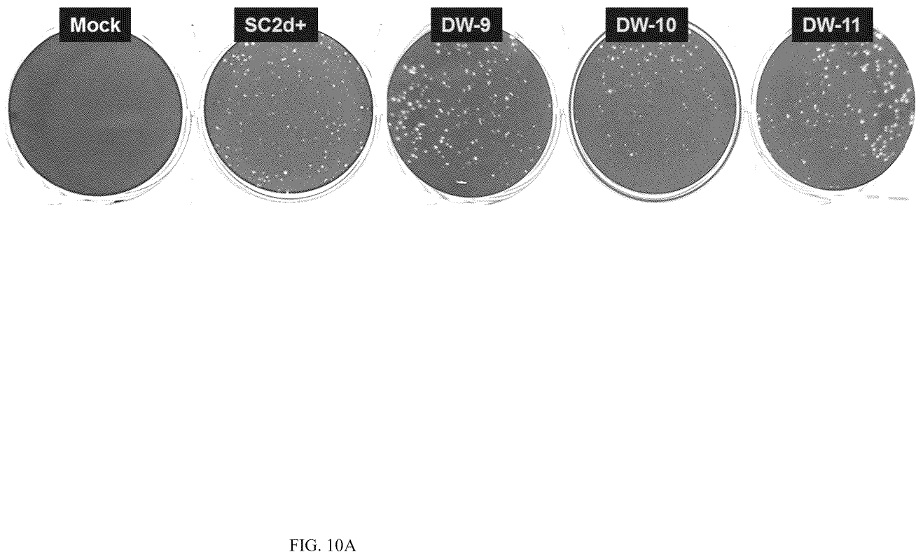

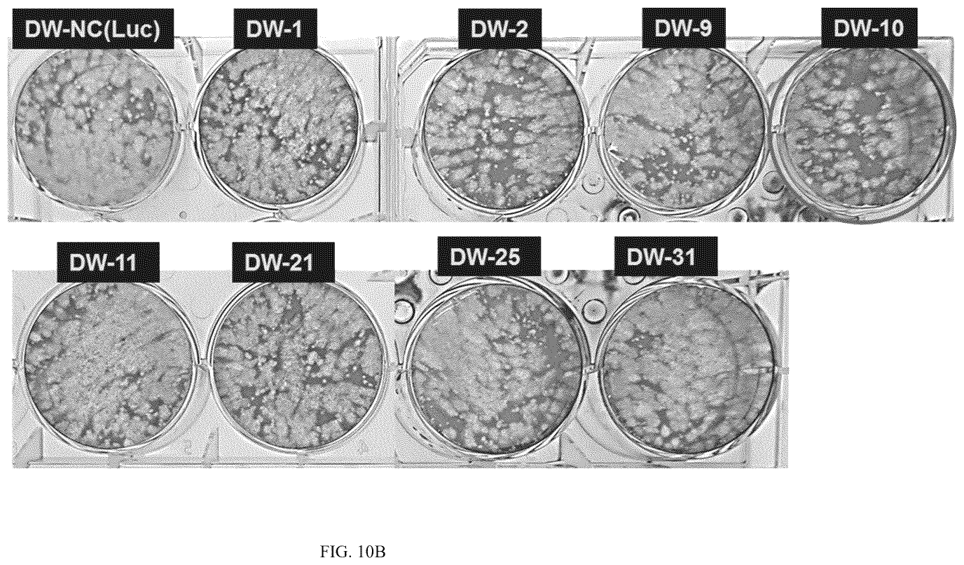

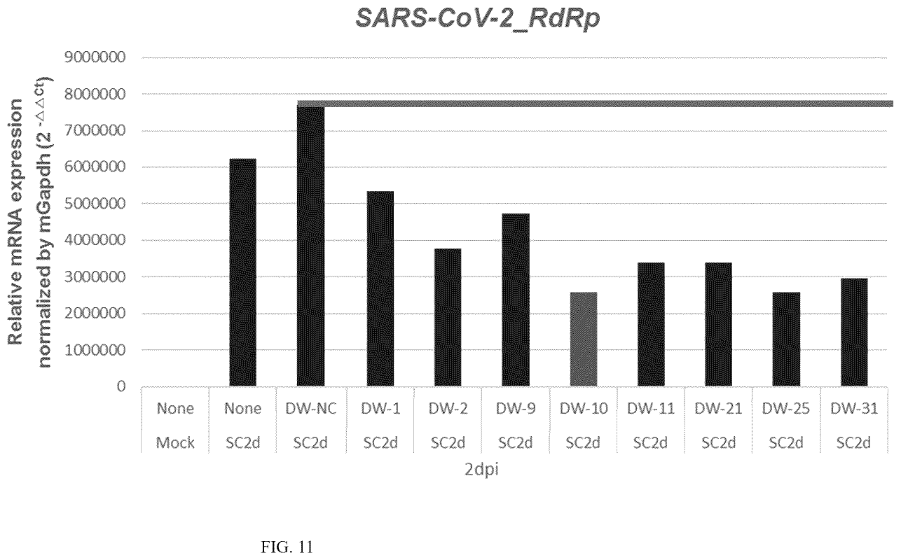

To evaluate the antiviral efficacy of eight antibody candidates, including DW-S-Ab-10, the candidates were applied either as pre-treatment [method \textcircled{1} below] or post-treatment [method \textcircled{2} below], followed by viral infection. Cellular efficacy was then assessed through cytopathic effect (CPE) analysis (FIGS. 8 and 9), plaque assays (FIGS. 10a and 10b), and qRT-PCR-based analysis of SARS-CoV-2 delta genes (FIG. 11).

\textcircled{1} Vero E6 cells were cultured in 6-well plates and pre-treated with LNP-formulated candidate mRNA antibody (mRNA-LNP). After 18 hours, the cells were infected with SARS-CoV-2 delta.

\textcircled{2} Vero E6 cells were cultured in 6-well plates and infected with SARS-CoV-2 delta, followed by treatment with the mRNA-antibody medium (mRNA: Ab medium) and subsequent incubation.

Cells were pre-treated with mRNA antibody-LNP for 18 hours, and cytopathic effects (CPE) were observed and imaged using an optical microscope 24 hours after viral infection. The cytopathic effect assay of the eight mRNA-antibody candidates demonstrated that DW-S-Ab-9 and DW-S-Ab-10 reduced cytopathic effects (FIG. 8).

In addition, cells were pre-treated with mRNA antibody-LNP for 18 hours, and cell viability (Relative Luminescence Units, RLU) was measured at 24 hours after viral infection using the CellTiter-Glo assay (Promega). The evaluation of cell viability and toxicity for the eight mRNA-antibody candidates showed that viral infection reduced cell viability due to cell damage, whereas treatment with DW-S-Ab-9 and DW-S-Ab-10 significantly increased cell viability. Furthermore, compared with the virus-infected group, cell viability was maintained without reduction, confirming the absence of cytotoxicity (FIG. 9).

Under conditions where cells were either pre-treated with mRNA antibody-LNP for 18 hours or treated with mRNA-antibody medium (mRNA: Ab medium) after viral infection, plaque assays were performed at 72 hours post-infection by fixing the cells with 4% paraformaldehyde (PFA), removing the agar, and staining with 0.2% crystal violet. The plaque assay evaluation of the eight mRNA-antibody candidates demonstrated that treatment with DW-S-Ab-10 resulted in reduced plaque formation in both pre-treatment and post-treatment conditions (FIGS. 10a-10b).

In addition, cells were pre-treated with mRNA antibody-LNP for 18 hours, harvested 48 hours after viral infection, and total RNA was extracted using the RNeasy Mini Kit (Qiagen). The SARS-CoV-2 delta RdRp gene was analyzed by quantitative (real-time) reverse transcription polymerase chain reaction (qRT-PCR). The results showed that DW-S-Ab-10 exhibited the greatest reduction (66.36%) in SARS-CoV-2 gene expression (FIG. 11).

### 3-4. Animal Efficacy Evaluation

All animal efficacy evaluation experiments were conducted under the approval of the Institutional Animal Care and Use Committee of the Agency for Defense Development (ADD-IACUC-22-16). Syrian Golden hamsters were used for the animal infection studies. Animals were maintained under controlled conditions at 22±3° C. temperatures, 55±15% relative humidity, and a 12-hour light/dark cycle. Standard rodent chow was provided ad libitum throughout the experimental period, and water from the municipal supply was freely available.

For the animal efficacy evaluation of antibody candidates including DW-S-Ab-10, the antibody medium (mRNA: Ab medium) was administered intranasally to hamsters at a dose of 1 mg per kg (mpk). After 24 hours, the animals were challenged with 500 plaque-forming units (PFU) of SARS-CoV-2 delta. At 5 days post infection (dpi), the animals were sacrificed, and lung tissues were subjected to gross examination and histopathological analysis using H&E staining.

The lung tissue damage scores for the mRNA-antibody candidates DW-S-Ab-10, DW-S-Ab-11, and DW-S-Ab-NC are presented in Table 5. According to Table 5, administration of DW-S-Ab-10 markedly reduced lung injury and inflammation, demonstrating superior efficacy compared to the control antibody Trastuzumab.

TABLE 5

| | | | | | | | | | | | |
|---|---|---|---|---|---|---|---|---|---|---|---|
| Virus | V-1 | 6 | DW-11 | 11-1 | 1.5 | DW-10 | 10-1 | 0.5 | DW-NC | T-1 | 3.5 |
| only | V-2 | 6 | | 11-2 | 1 | | 10-2 | 1.0 | (Trastuzumab) | T-2 | 1.0 |
| | V-3 | 6 | | 11-3 | 1 | | 10-3 | 3.0 | | T-3 | 4.5 |
| | V-4 | 3 | | 11-4 | 5 | | 10-4 | 0.5 | | T-4 | 2.5 |
| | V-5 | 3 | | 11-5 | 6 | | 10-5 | 2.0 | | T-5 | 4.0 |
| | Mean | 4.8 | | Mean | 2.7 | | Mean | 1.4 | | Mean | 3.1 |
| | Standard | 1.643 | | Standard | 2.100 | | Standard | 1.083 | | Standard | 1.387 |
| | Deviation | 168 | | Deviation | 502 | | Deviation | 974 | | Deviation | 444 |

*Lung injury score scale: 0-10 points, 10 points indicating damage across the entire lung area, and 0 points indicating no lung injury.

Referring to the lung tissue images of the mRNA-antibody candidates in FIG. 12a, it was confirmed that lung injury in the DW-S-Ab-10 administration group was significantly reduced compared with the SARS-CoV-2 delta virus-infected group (SC2d+). In addition, referring to FIG. 12b, it was confirmed that lung injury and inflammation were decreased in the DW-S-Ab-10 administration group.

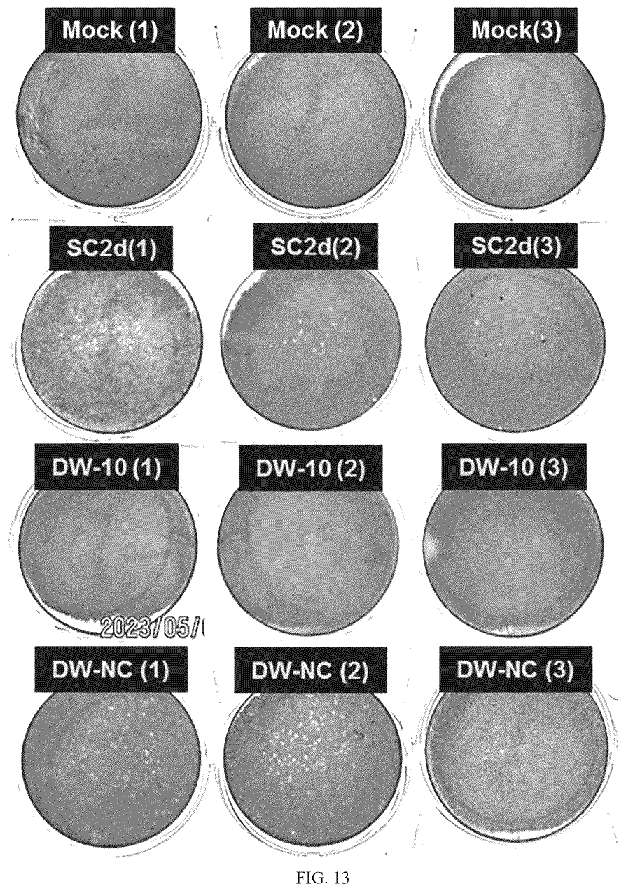

For the in vivo efficacy evaluation of DW-S-Ab-10, lung tissue homogenates from hamsters subjected to viral infection and administration of the therapeutic candidate were used to infect Vero E6 cells, and viral titers in the tissue were determined by plaque assay (FIG. 13).

Referring to FIG. 13, it was confirmed that plaques were reduced in the DW-S-Ab-10 administration group compared with the virus-infected group.

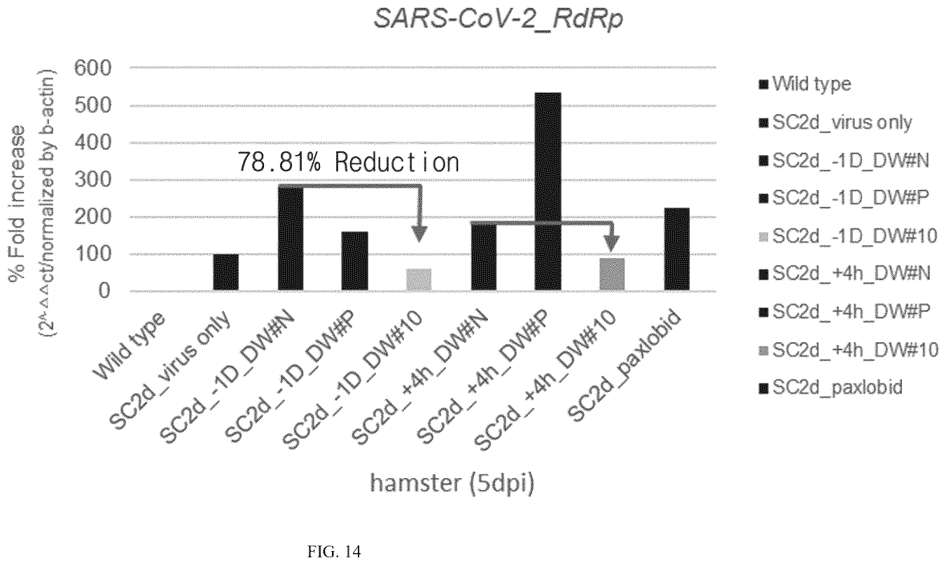

In addition, an animal efficacy evaluation including the conventional drug Paxlovid was performed in the same manner, wherein total RNA was extracted from the lung tissue homogenates using the RNeasy Mini Kit (Qiagen), and viral genes were analyzed by qRT-PCR (FIG. 14).

Referring to FIG. 14, it was confirmed that the SARS-CoV-2 gene was reduced by up to 78.81% in the DW-S-Ab-10 administration group compared with the virus-infected group. Furthermore, while the therapeutic candidate of the present disclosure was administered only once, Paxlovid was administered four times; nevertheless, the viral titer in the DW-S-Ab-10 single-dose group was relatively lower than that in the four-dose Paxlovid group, thereby demonstrating that the therapeutic candidate of the present disclosure exhibits markedly superior efficacy compared with conventional Paxlovid.

In the above, exemplary embodiments of an mRNA antibody for treating SARS-CoV-2 Delta infection and a composition comprising the same according to the present disclosure have been described. Moreover, it will be appreciated that various modifications to these exemplary embodiments are possible without departing from the scope of the present disclosure.

The scope of the present disclosure should therefore not be limited to those exemplary embodiments described above, but should be defined by the following claims and their equivalents.

In other words, the foregoing exemplary embodiments are to be understood as illustrative rather than restrictive in all respects, and the scope of the present disclosure is indicated by the following claims rather than the detailed description. All modifications or variations derived from the meaning, scope, and equivalent concepts of the claims should be interpreted as being included within the scope of the present disclosure.

### INDUSTRIAL APPLICABILITY

The present disclosure provides an mRNA antibody for treating SARS-CoV-2 Delta infection and a composition comprising the same.

### SEQUENCE LISTING

SEQ ID NO: 1 is the mRNA sequence of the heavy chain of antibody candidate DW-S-Ab-01.

SEQ ID NO: 2 is the amino acid sequence of the heavy chain of antibody candidate DW-S-Ab-01.

SEQ ID NO: 3 is the mRNA sequence of the heavy chain of antibody candidate DW-S-Ab-02.

SEQ ID NO: 4 is the amino acid sequence of the heavy chain of antibody candidate DW-S-Ab-02.

SEQ ID NO: 5 is the mRNA sequence of the heavy chain of antibody candidate DW-S-Ab-09.

SEQ ID NO: 6 is the amino acid sequence of the heavy chain of antibody candidate DW-S-Ab-09.

SEQ ID NO: 7 is the mRNA sequence of the heavy chain of antibody candidate DW-S-Ab-10.

SEQ ID NO: 8 is the amino acid sequence of the heavy chain of antibody candidate DW-S-Ab-10.

SEQ ID NO: 9 is the mRNA sequence of the heavy chain of antibody candidate DW-S-Ab-11.

SEQ ID NO: 10 is the amino acid sequence of the heavy chain of antibody candidate DW-S-Ab-11.

SEQ ID NO: 11 is the mRNA sequence of the heavy chain of antibody candidate DW-S-Ab-21.

SEQ ID NO: 12 is the amino acid sequence of the heavy chain of antibody candidate DW-S-Ab-21.

SEQ ID NO: 13 is the mRNA sequence of the heavy chain of antibody candidate DW-S-Ab-25.

SEQ ID NO: 14 is the amino acid sequence of the heavy chain of antibody candidate DW-S-Ab-25.

SEQ ID NO: 15 is the mRNA sequence of the heavy chain of antibody candidate DW-S-Ab-31.

SEQ ID NO: 16 is the amino acid sequence of the heavy chain of antibody candidate DW-S-Ab-31.

SEQ ID NO: 17 is the mRNA sequence of the light chain of antibody candidate DW-S-Ab-01.

SEQ ID NO: 18 is the amino acid sequence of the light chain of antibody candidate DW-S-Ab-01.

SEQ ID NO: 19 is the mRNA sequence of the light chain of antibody candidate DW-S-Ab-02.

SEQ ID NO: 20 is the amino acid sequence of the light chain of antibody candidate DW-S-Ab-02.

SEQ ID NO: 21 is the mRNA sequence of the light chain of antibody candidate DW-S-Ab-09.

SEQ ID NO: 22 is the amino acid sequence of the light chain of antibody candidate DW-S-Ab-09.

SEQ ID NO: 23 is the mRNA sequence of the light chain of antibody candidate DW-S-Ab-10.

SEQ ID NO: 24 is the amino acid sequence of the light chain of antibody candidate DW-S-Ab-10.

SEQ ID NO: 25 is the mRNA sequence of the light chain of antibody candidate DW-S-Ab-11.

SEQ ID NO: 26 is the amino acid sequence of the light chain of antibody candidate DW-S-Ab-11.

SEQ ID NO: 27 is the mRNA sequence of the light chain of antibody candidate DW-S-Ab-21.

SEQ ID NO: 28 is the amino acid sequence of the light chain of antibody candidate DW-S-Ab-21.

SEQ ID NO: 29 is the mRNA sequence of the light chain of antibody candidate DW-S-Ab-25.

SEQ ID NO: 30 is the amino acid sequence of the light chain of antibody candidate DW-S-Ab-25.

SEQ ID NO: 31 is the mRNA sequence of the light chain of antibody candidate DW-S-Ab-31.

SEQ ID NO: 32 is the amino acid sequence of the light chain of antibody candidate DW-S-Ab-31.

1. (canceled)

2. (canceled)

3. An mRNA antibody for treating SARS-CoV-2 Delta infection, comprising the nucleotide sequence of SEQ ID NO: 7 and the nucleotide sequence of SEQ ID NO: 23.

4. (canceled)

5. (canceled)

6. A lipid nanoparticle (LNP) composition for treating SARS-CoV-2 Delta infection, comprising the mRNA of claim 1.

7. A recombinant expression vector comprising the mRNA of claim 1.

8. An mRNA antibody composition for treating SARS-CoV-2 Delta infection, comprising:
a lipid nanoparticle (LNP) composition of claim 2; and
a pharmaceutically acceptable carrier.

9. (canceled)
